(19) United States
(12) Patent Application Publication
(10) Pub. No.: US 2004/0133130 A1
(43) Pub. Date: Jul. 8, 2004

(54) Magnetically navigable medical guidewire

(76) Inventors: Steven J. Ferry, Excelsior, MN (US); Roger N. Hastings, Maple Grove, MN (US); Reed A. Houge, Buffalo, NY (US)

Correspondence Address: HARNESS, DICKEY, & PIERCE, P.L.C, 7700 BONHOMME, STE 400, ST. LOUIS, MO, 63105, US

(21) Appl. No.: 10/337,236

(22) Filed: Jan. 6, 2003

(51) Int. Cl. A61B 5/00; A61M 25/00

(52) U.S. Cl. 600/585

(57) ABSTRACT

A magnetically navigable medical guidewire, comprising an elongate wire having a proximal end, and a distal end; a hollow cylinder secured on the distal end of the wire forming the tip of the guidewire. A magnetically responsive element is disposed inside said hollow cylinder. The distal tip has a rounded, dome-shaped configuration.

### BACKGROUND OF THE INVENTION

Medical guidewires are used to facilitate the navigation of medical devices into branches in a subject's vasculature. Conventional guidewires either have a permanent bend, e.g., ay “J” formed in their distal tip, or are constructed so that the user can form the distal tip in the a desired configuration. The tip of the guidewire is advanced to location adjacent the branch that the user wants it to enter, and the proximal end of the guidewire is repeatedly torqued to rotate the distal tip while the wire is pushed. This action is repeated until, by trial and error, the tip enters the desired vessel branch. The repeated twisting and advancing of the tip against the vessel wall can scratch or abrade the wall of the vessel. Where the guidewire has passed through several bends, the guidewire may contact the vessel wall at several points along its length, and twisting the guidewire can abrade the vessel wall at each of these points of contact. Moreover, after the guidewire has made several bends, the guidewire becomes increasingly difficult to control, requiring repeated attempts to enter a desired vessel branch. This trial and error method can frustrate the physician and cause additional wall contact and potential trauma.

To address these and other difficulties, magnetically navigable guidewires have been developed which can be controlled with the application of an external magnetic field. An example of magnetically navigable guidewire is disclosed in Werp et al., U.S. Pat. No. 5,931,818 (incorporated in its entirety herein by reference). The user can advance the magnetically navigable guide wire into vessels with little or no contact between the end of the wire and the vessel wall. When the distal end of the guidewire is adjacent the branch of interest, the user operates a magnetic system to apply a magnetic field (with the aid of a computerized user interface) to deflect the wire tip into the vessel side branch. The magnet system can be made sufficiently accurate to direct the distal end of the guidewire into the brach on the first effort, eliminating the trial and error of manually operated guidewires and thereby reducing or eliminating trauma to the vessel wall. A single guide wire can be used for all turns, so the wire never needs to be exchanged, saving time and cost. The wire can be navigated alone, without the support of an adjacent catheter, regardless of the number of turns the wire has already made. This is because deflection of the guidewire tip is controlled by the external magnets and is independent of the proximal wire path. Tip torque response is irrelevant in magnetic navigation, and in normal use, the physician does not apply torque to the guidewire.

### SUMMARY OF THE INVENTION

The present invention relates to magnetically navigable medical guidewires, and in particular to improvements in the construction of such guidewires. Generally, a guidewire constructed in accordance with the principles of this invention comprises: an elongate wire having a proximal end and a distal end. There is a radioopaque sleeve at the distal end of the wire. A magnetically responsive element is sealed in the radiopaque sleeve. This magnetically responsive element preferably comprises a permanent magnetic material, but may alternatively comprise a permeable magnetic material. In addition, the guidewire can include a permeable magnetic material proximal to the magnetically responsive element. This magnetic material may be a coil surrounding the wire proximal to the sleeve.

### BRIEF DESCRIPTION OF THE DRAWINGS

FIG. 1 is a side elevation view of a preferred embodiment guidewire constructed according to the principles of this invention;

FIG. 2 is a side elevation view of the core wire comprising the guidewire;

FIG. 3 is a enlarged partial side elevation view of the core wire showing the proximal stage for mounting a coil in the preferred embodiment of this invention;

FIG. 4 is a enlarged partial side elevation view of the core wire showing the distal stage for mounting a coil in the preferred embodiment of this invention;

FIG. 5 is a side elevation view of the guidewire with the distal tip shown in longitudinal cross section;

FIG. 6 is a partial longitudinal cross-sectional view of the core wire showing the proximal stage for mounting a coil in the preferred embodiment of this invention;

FIG. 7 is a partial longitudinal cross-section view of the core wire showing the distal stage for mounting a coil in the preferred embodiment of this invention;

FIG. 8 is an enlarged cross sectional view of the distal tip of an alternate construction of the guide wire of the preferred embodiment;

FIG. 9A is side elevation view of the distal tip cap used in the the alternate construction of the guide wire;

FIG. 9B is a longitudinal cross-sectional view of the distal tip cap, taken along the plane of line 9B-9B in FIG. 9A; and

FIG. 10 is a diagram showing the bending of the distal end of the guidewire in an applied magnetic field.

Corresponding reference numerals indicate corresponding parts throughout the several views of the drawings.

### DETAILED DESCRIPTION OF THE INVENTION

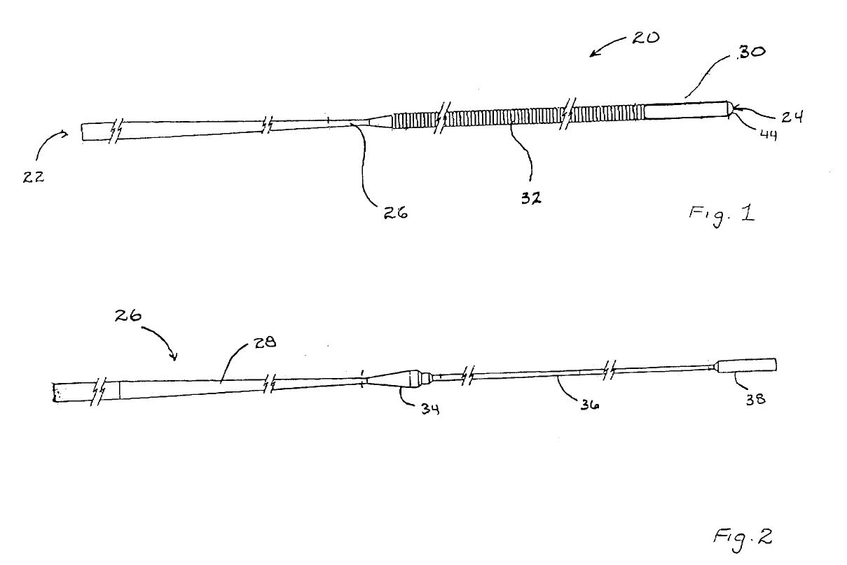

A first preferred embodiment of a magnetically navigable medical guidewire is indicated generally as 20 in FIG. 1. The guidewire 20 has a proximal end 22 and a distal end 24 and comprises a flexible core wire 26 extending from the proximal end substantially to the distal end. The core wire 26 can be made of Nitinol or other suitable material. As shown in FIG. 2, the core wire 26, or preferably the distal end section 28 of the core wire, preferably tapers so that the flexibility of the distal end section of the guidewire 20 generally increases toward the distal end. However, the flexibility does not necessarily increase continuously, and for example may have sections of increasing flexibility and sections of constant flexibility, or may comprises a plurality of sections of constant flexibility, where each section is more flexible than the next most proximal. Furthermore, rather than tapering the wire, the increasing flexibility can be provided in some other way, for example by using different materials or different heat treatments, etc.

As described in more detail below, a magnetically responsive element 30 is provided on the distal end of the wire 22. This element 30 can include a permanent magnetic material or a permeable magnetic material, and is of sufficient size and shape to cause the distal end portion of the guidewire 20 to align in a selected direction with a magnetic field applied from an external source magnet. The guidewire 20 is sufficiently stiff that it can be advanced in the selected direction by pushing the proximal end of the guidewire 20.

As also described in more detail below, a coil 32 is preferably mounted over a distal end portion of the guidewire 20, to resist kinking as the distal tip bends in response to an applied magnetic field.

In this preferred embodiment, a proximal stage 34 is formed at the distal end of the distal end section 28 of the core wire 26, for mounting the proximal end of the coil 32. As shown best in FIG. 3, the stage 34 comprises a first section 34a that widens the distal direction, a second section 34b of generally constant cross-section, a third section 34c that tapers in the distal direction, a fourth section 34d of generally constant cross section, and a fifth section 34e that tapers in the distal direction. Immediately distal to the proximal stage 34, the guide wire has a distal tip section 36, which terminates in a distal stage 38, that mounts the distal end of the coil 32, and also helps mount the magnetically responsive element 30. As best shown in FIG. 4, the distal stage 38 comprises a first section 38a that widens in the distal direction, and second section 38b of generally constant cross section.

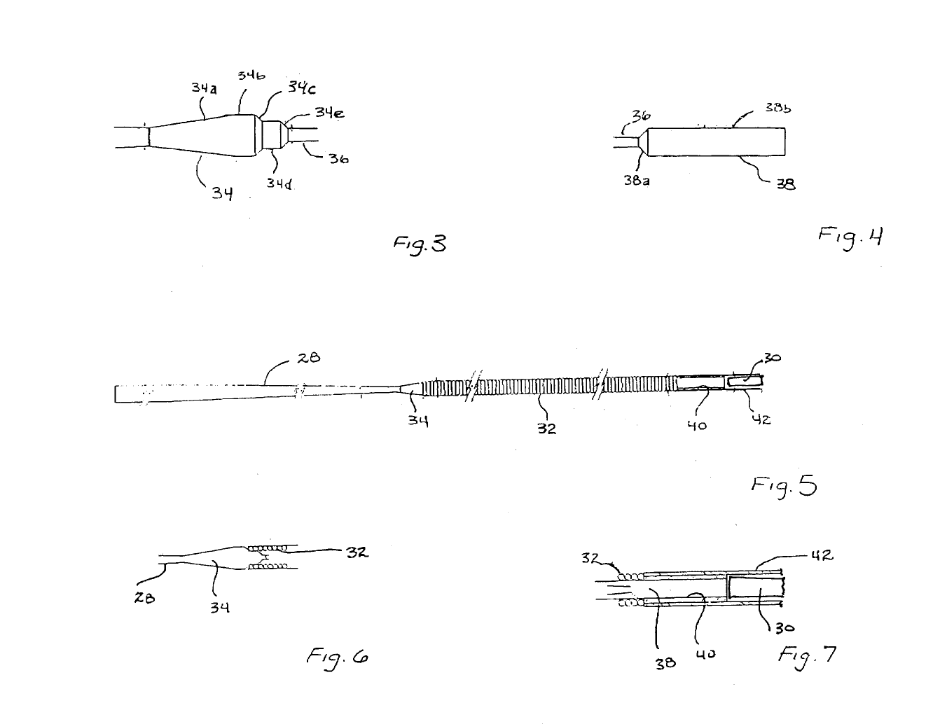

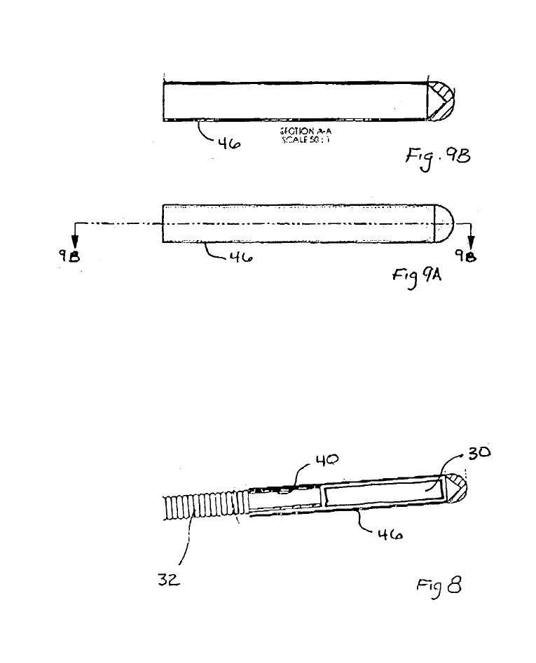

As shown in FIG. 6, the proximal end of the coil 32 is mounted on the section 34d of the proximal stage 34, and as shown in FIG. 7, the distal end of the coil 32 is mounted on the proximal end of section 38b of the distal stage 38, and the ends of the coil are secured to their respective stages as by welding. The coil 32 is preferably made of a wire of a magnetically permeable material, such as Hiperco. In this preferred embodiment, the wire is a 0.002 inch diameter Hiperco wire.

A collar 40 is mounted over the distal end of section 38b of the distal stage 38, and is secured thereto by welding. In the preferred embodiment, the proximal end of a sleeve 42 is mounted over the collar 40, and secured thereto as by welding. The distal end of the sleeve projects beyond the distal end of the stage 38, forming a cylindrical socket for receiving the magnetic element 30. The magnetic element 30 is secured therein, for example with a bead of epoxy 44, which forms a smooth, rounded, dome shape at the distal end of the guidewire 20 to resist scratching and abrasion of the vessel walls.

The sleeve 42 is preferably made of a or at least plated with, radiopaque material so that the distal end of the guidewire 20 can be seen in x-ray imaging. The sleeve 42 may be made of gold, a gold alloy, platinum, platinum iridium; or other platinum alloy. The magnetically responsive element 30, which can be made of a permanent magnetic material or a permeable magnetic material, is disposed inside the sleeve 42. Suitable permanent magnetic materials include neodymium-iron-boron (Nd—Fe—B). Suitable permeable magnetic materials include magnetic stainless steel, such as a 303 or 304 stainless steel, Hiperco. The size and material of the magnetically responsive element and flexibility of the distal end portion of the wire 22 are selected so that the distal end portion of the guide wire can be reoriented by the application of a magnetic field of no more than about 0.15 Tesla, and more preferably no more than about 0.10 Tesla.

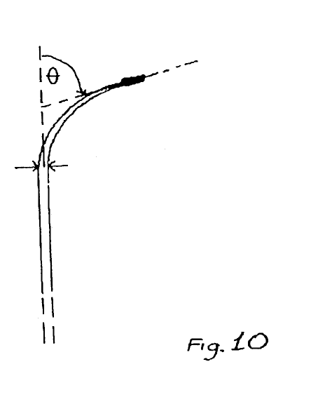

An alternate construction of this preferred embodiment is shown in FIG. 8. As shown in FIG. 8, instead of a sleeve 42, a cup 44 having a rounded end, is secured over the collar 40, with the magnetic element 30 enclosed therein. The cup 44 is shown in more detail in FIGS. 9A and 9B, and can be made of the same material as the sleeve 42.

By way of example only, and without limiting the invention the guidewire of the preferred embodiment has a total length of about 13 inches. The distal section 28 is 11.81 inches long, and tapers from a thickness (diameter) of about 0.14 inches to about 0.0049 inches. The first section 34a widens from a thickness of about 0.049 inches to about 0.138 inches over a length of 0.045 inches the second section has a thickness of about 0.0138 inches and a length of 0.010 inches; the third section 34c tapers from a thickness of about 0.0138 inches to about 0.0088 inches over a length of about 0.003 inches the fourth section 34d has a thickness of about 0.0088 inches and a length of about 0.015 inches and the fifth section 34e tapers from a thickness of 0.0088 inches to a thickness of 0.0039 over a length of 0.003 inches. The first section 38a widens from a thickness of 0.0039 inches to a thickness of 0.0088 inches over a length of 0.003 inches; and the section 38b has a thickness of 0.0088 inches and a length of 0.250 inches. The distal tip section 36 tapers from a thickness of about 0.0039 inches at the proximal end, to a thickness of about 0.0025 inches, at the distal end, over a length of about 0.788 inches.

The magnetic coupling between the tip magnet and externally applied magnetic fields must be sufficient to overcome the restoring torque of the guidewire to provide adequate deflection. It is known in the art that when a wire is held at a distance “L” proximal to its tip, the angle of deflection is given by:

θ=θ₀ sin(Δ) (1)

where θ=deflection angle of tip relative to the body of the wire (see FIG. 10)

θ₀=maximum angle of deflection of tip relative to wire body

Δ=angle between the tip magnet and the applied magnetic field

The maximum tip deflection angle occurs when the applied field is at right angles to the tip magnet (Δ=90°) or sin(Δ)=1 in Eq.(1). The tip deflection angle is shown in FIG. 10.

The maximum deflection angle, θ₀, is given by:

θ₀=32mHL/(πYd⁴) (2)

where m=tip magnet magnetic moment in A m²

H=applied magnetic field in Tesla

L=free length of wire (distal to pinning point) in m.

Y=Young's modulus in N/m²

d=wire diameter in m.

From Eq. (2) it is seen that for a given wire diameter, the deflection angle scales with the free length of wire at the distal tip. Guidewire deflection can be experimentally measured and compared to Eq. (2) by holding the wire at a set distance proximal to the tip, and applying a magnetic field of known magnitude, H, at varying angles to the tip until the maximum tip deflection is observed (which occurs when the field is at right angles to the tip).

In guidewire navigation through blood vessels, the point at which the wire is “held” depends upon the vessel diameter and curvature. A representative free length of 0.5 inches has been chosen for definiteness in laboratory testing. This free length produces deflection angles that are typical of angles seen in animal and human vessel navigation.

Guidewire performance is judged in the laboratory by the deflection angle achieved in a given applied magnetic field when the free length (distance form wire tip to pinning point) is 0.5 inches. For example, in the Stereotaxis Niobe™ magnetic navigation system, an external field of 0.1 Tesla can be applied within the patient in any direction. The maximum deflection angle of the guidewire in a 0.1 Tesla field is thus one way to characterize the wire performance in the Niobe™ magnetic navigation system.

Tip deflection angle required for vessel navigation is learned through experience. Arenson et al., U.S. Pat. No. 6,304,769 (incorporated herein by reference) suggests that a tip deflection angle as small as 6 degrees is adequate for magnetic navigation of a catheter. However, the inventors believe, based upon a collective and representative view of physicians who have used the Stereotaxis' magnetic navigation system in animal and human blood vessels, that 50 degrees of tip deflection is required to be able to access the majority of vessel branches. The inventors have determined that a minimum tip deflection of about 30 degrees is required for navigation, that a minimum tip deflection of about 50 degrees is desirable, and that larger angles, between about 75 and about 90 degrees, are preferred.

What is claimed is:

1. A magnetically navigable medical guidewire, comprising an elongate wire having a proximal end, and a distal end; a hollow cylinder secured on the distal end of the wire forming the tip of the guidewire; a magnetically responsive element inside said hollow cylinder; and a dome-shaped cap securing the magnetically responsive element inside the hollow cylinder.

2. The magnetically navigable medical guidewire according to claim 1 wherein the flexibility of the guidewire increases toward the distal end.

3. The magnetically navigable medical guidewire according to claim 1 wherein the hollow cylinder is radioopaque.

4. The magnetically navigable medical guidewire according to claim 3 wherein the radiopaque hollow cylinder is made of gold or a gold alloy.

5. The magnetically navigable medical guidewire according to claim 3 wherein the radiopaque hollow cylinder is made of platinum or a platinum alloy.

6. The magnetically navigable medical guidewire according to claim 1 wherein the dome-shaped cap comprises a settable epoxy, closing the mouth of the hollow cylinder.

7. The magnetically navigable medical guidewire according to claim 1 wherein the hollow cylinder is closed at its distal end, forming a hollow cylinder with a dome-shaped distal tip.

8. The magnetically navigable guidewire according to claim 1 in which the hollow cylinder containing the magnetically responsive material is welded at its proximal end to the distal end of the wire.

9. The magnetically navigable medical guidewire according to claim 1 wherein the magnetically responsive element comprises a permeable magnetic material.

10. The magnetically navigable medical guidewire according to claim 1 wherein the magnetically responsive element comprises a permanent magnetic material.

11. The magnetically navigable medical guidewire according to claim 1 wherein the magnetically responsive element comprises a permanent magnetic material, and wherein a portion of the guide wire proximal to the magnetically responsive element is formed of a permeable magnetic material.

12. The magnetically navigable medical guidewire according to claim 1 wherein the magnetically responsive element comprises a permanent magnetic material, and further comprising a coil of a permeable magnetic material surrounding the distal end portion of the guidewire, proximal to the magnetically responsive element.

13. The magnetically navigable medical guidewire according to claim 1 wherein the magnetically responsive element and the stiffness of the distal end portion of the wire are such that, when the guidewire is held at a point 0.5 inches proximal to its distal tip, the maximum angle of deflection of the guidewire tip relative to the body of the guidewire is at least 30 degrees when the applied magnetic field has a magnitude of at least 0.1 Tesla.

14. The magnetically navigable medical guidewire according to claim 13 wherein the magnetically responsive element and the stiffness of the distal end portion of the wire are such that, when the guidewire is held at a point 0.5 inches proximal to its distal tip, the maximum angle of deflection of the guidwire tip relative to the body of the guidewire is at least 30 degrees when the applied magnetic field has a magnitude of at least 0.05 Tesla.

15. A magnetically navigable medical guidewire, comprising an elongate wire having a proximal end, and a distal end; a hollow cup having a generally cylindrical sidewall, and a closed bottom on the distal end of the wire; and a magnetically responsive element disposed inside said cup.

16. A magnetically navigable medical guidewire according to claim 15, wherein the cup is made of a radioopaque material.

17. The magnetically navigable medical guidewire according to claim 16, wherein the hollow cylinder is made from gold or a gold alloy.

18. The magnetically navigable medical guidewire according to claim 15 wherein the magnetically responsive element comprises a permanent magnetic material.

19. The magnetically navigable medical guidewire according to claim 15 wherein the magnetically responsive element and the stiffness of the distal end portion of the wire are such that, when the guidewire is held at a point 0.5 inches proximal to its distal tip, the maximum angle of deflection of the guidewire tip relative to the body of the guidewire is at least 30 degrees when the applied magnetic field has a magnitude of at least 0.1 Tesla.

20. The magnetically navigable medical guidewire according to claim 19 wherein the magnetically responsive element and the stiffness of the distal end portion of the wire are such that, when the guidewire is held at a point 0.5 inches proximal to its distal tip, the maximum angle of deflection of the guidewire tip relative to the body of the guidewire is at least 30 degrees when the applied magnetic field has a magnitude of at least 0.05 Tesla.

21. The magnetically navigable medical guidewire according to claim 15 wherein the magnetically responsive element comprises a permanent magnetic material, and further comprising a coil of a permeable magnetic material surrounding the distal end portion of the guidewire, proximal to the magnetically responsive element.

23. The magnetically navigable medical guidewire according to claim 1 wherein the magnetically responsive element comprises a permanent magnetic material, and further comprising a coil of a permeable magnetic material surrounding the distal end portion of the guidewire, proximal to the magnetically responsive element.
